(19) United States
(12) Patent Application Publication
(10) Pub. No.: US 2005/0234507 A1
(43) Pub. Date: Oct. 20, 2005

(54) Medical tool for access to internal tissue

(76) Inventors: Jeff Geske, Fridley, MN (US); Steven L. Waldhauser, Circle Pines, MN (US); Kenneth C. Gardeski, Plymouth, MN (US)

Correspondence Address: MEDTRONIC, INC., 710 MEDTRONIC PARKWAY NE, MS-LC340, MINNEAPOLIS, MN, 55432-5604, US

(21) Appl. No.: 11/096,672

(22) Filed: Mar. 31, 2005

Related U.S. Application Data
(60) Provisional application No. 60/563,023, filed on Apr. 16, 2004.

(51) Int. Cl. A61B 17/28

(52) U.S. Cl. 606/207; 606/129; 600/217; 606/108; 600/218; 604/93.01

(57) ABSTRACT

A medical tool for minimally invasive access to the pericardium or similar internal tissue. The medical tool includes a shaft with jaws disposed on a distal end of the shaft and a mechanism for opening and closing the jaws connected to the proximal end of the shaft. The jaws include needle-like elements that extend from the jaws. A delivery element defining a delivery lumen extends along the tool, parallel and adjacent to the shaft, with a distal end of the delivery element being positioned between the jaws. Moreover, the distal ends of each of the jaws define a semi-circular shape such that when the first and second jaws are closed, a hole through the closed jaws is formed by the semi-circular shapes.

### FIELD OF THE INVENTION

The invention relates to medical tools and, more particularly, medical tools that facilitate minimally invasive access to the pericardium or other similar internal tissue.

### BACKGROUND OF THE INVENTION

The pericardium (also referred to at the pericardial sac) is a conical membranous sac that contains the heart and the great vessels that surround the heart. The pericardium is fluid-filled and functions to prevent dilation of the heart chambers. The pericardium also lubricates the surfaces of the heart and maintains the heart in a fixed geometric position. In addition, the pericardium provides a barrier that prevents the spread of infection from adjacent structures in the chest cavity and prevents surrounding tissue from adhering to the heart.

The space between the pericardium and the heart is referred to as the pericardial space. The pericardial space contains the pericardial fluid, and is relatively small in volume. In some cases, it is desirable to access the pericardial space for diagnostic or therapeutic purposes. In particular, pericardiocentesis, or puncture of the pericardium, has been performed for a number or purposes, including diagnosis of pericardial disease by study of the pericardial fluid, withdrawal of pericardial fluid for treatment of acute cardiac tamponade, infusion of therapeutic agents or drugs, and other purposes. Tools for minimally invasive access to the pericardium and similar internal tissue are highly desirable, whether subxiphoid or transthoracic.

### BRIEF SUMMARY OF THE INVENTION

In general, the invention is directed to a medical tool for minimally invasive access to the pericardium or similar internal tissue. The medical tool includes a shaft with jaws disposed on a distal end of the shaft and a mechanism for opening and closing the jaws connected to the proximal end of the shaft. In order to facilitate grasping of the pericardium, the jaws include needle-like elements that extend from the jaws. A delivery element defining a delivery lumen extends along the tool, parallel and adjacent to the shaft, with a distal end of the delivery element being positioned between the jaws. Moreover, the distal ends of each of the jaws define semi-circular shapes, e.g., grooves, such that when the first and second jaws are closed, a hole through the closed jaws is formed by the semi-circular shapes. The distal end of the delivery element passes through the hole in the closed jaws to facilitate access to the pericardium when the pericardium is grasped by the jaws.

In one embodiment, the invention provides a medical tool that includes a shaft defining a proximal end and a distal end, and first and second jaws disposed on the distal end of the shaft. A first needle-like element protrudes from the first jaw and a second needle-like element protrudes from the second jaw. A delivery element defining a delivery lumen is positioned parallel and adjacent to the shaft with a distal end of the delivery element being disposed between the first and second jaws. A mechanism is coupled to the proximal end of the shaft and mechanically coupled to the first and second jaws to open and close the first and second jaws.

In another embodiment, the invention provides a pericardial access tool that includes a shaft defining a proximal end and a distal end and first and second jaws disposed on the distal end of the shaft. First and second needle-like elements protrude respectively from the first and second jaws for puncturing a pericardium so that the first and second jaws can grasp the pericardium. In addition, the tool includes a delivery means positioned parallel and adjacent to the shaft with a distal end of the delivery means being disposed between the first and second jaws for insertion into the pericardium when the pericardium is grasped by the first and second jaws. Also, the tool includes a mechanism coupled to the proximal end of the shaft and mechanically coupled to the first and second jaws to open and close the first and second jaws in order to grasp the pericardium. For example, the mechanism may include a trigger, button, or the like, that can be actuated to open and close the jaws of the tool.

In another embodiment, the invention provides a method that includes selecting a size of needle-like elements of a pericardial access tool based on inspection of a pericardium of a patient, inserting the pericardial access tool into a patient, the pericardial access tool including needle-like elements of the selected size disposed on jaws of the tool, and grasping the pericardium with the jaws of the tool.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

These and other advantages and features of the present invention will be appreciated as the same becomes better understood by reference to the following detailed description of the preferred embodiment of the invention when considered in connection with the accompanying drawings, in which like numbered reference numbers designate like parts throughout the figures thereof, and wherein:

FIG. 1 is a conceptual view illustrating a patient and a medical tool according to an embodiment of the invention.

FIG. 2 is a side view of an exemplary medical tool according to an embodiment of the invention.

FIG. 3 is a perspective view of an exemplary medical tool according to another embodiment of the invention.

FIGS. 4 and 5 are top views of a distal end of a medical tool according to an embodiment of the invention.

FIGS. 6A and 6B are front views of a distal tip of a medical tool according to an embodiment of the invention.

FIGS. 7-9 are perspective views of a distal tip of a medical tool according to an embodiment of the invention.

FIG. 10 is a flow diagram according to an embodiment of the invention, illustrating a minimally invasive medical procedure to provide therapy to a pericardium of a patient.

FIGS. 11A and 11B are conceptual views of a distal end of a medial tool grasping the pericardium of a patient's heart in accordance with an embodiment of the invention.

### DETAILED DESCRIPTION OF THE INVENTION

The invention is directed to a medical tool for minimally invasive access to the pericardium or similar internal tissue, such as the dura of the brain, a spinal membrane, a thoracic membrane, the pleural lining of the lungs, and so forth. The pericardium is a conical membranous sac that contains the heart and the great vessels that surround the heart. For certain patients, it can be desirable to access the pericardial space for diagnostic or therapeutic purposes. Accordingly, tools for minimally invasive access to the pericardium are highly desirable.

In accordance with the invention, the medical tool includes a shaft with jaws disposed on a distal end of the shaft, and a mechanism for opening and closing the jaws connected to the proximal end of the shaft. In order to facilitate grasping of the pericardium, the jaws include needle-like elements that extend from the jaws. A delivery element defining a delivery lumen extends along the tool, parallel and adjacent to the shaft, with a distal end of the delivery element being positioned between the jaws. The distal ends of each of the jaws define a semi-circular shape such that when the first and second jaws are closed, a hole through the closed jaws is formed by the semi-circular shapes. For example, a needle or other structure may can be passed through the delivery element and through the hole formed by the closed jaws in order to facilitate access to the pericardium when the pericardium is grasped by the jaws.

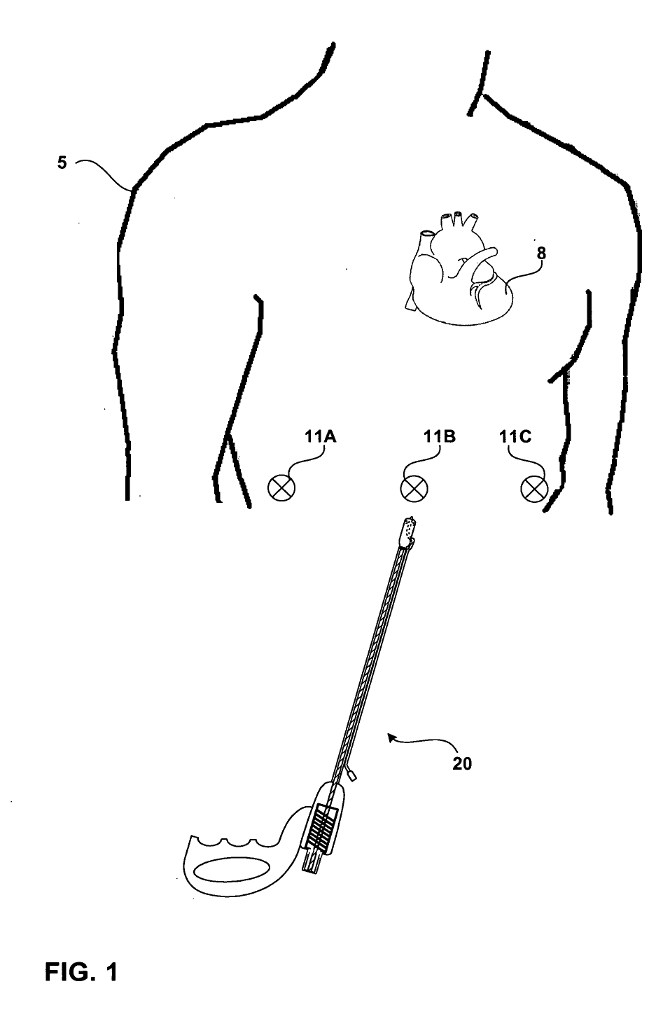

FIG. 1 is a conceptual view of a medical tool 20 that can be used for minimally invasive access to a pericardium of patient 5. Numerous other uses of medical tool 20, however, also exist. Medical tool 20 is inserted into a patient 5, e.g., through one of locations 11A, 11B or 11C, or possibly through the ribs of patient 5 in a thoracotomy-like approach for access to the pericardium of heart 8 of patient 5. More specifically, the procedure involves puncture of patient 5 and insertion of an introducer, such as a catheter, or the like. The introducer is directed to a target site, such as to the pericardium of heart 8. Medical tool 20 is then inserted through the introducer for access to the pericardium. A number of features of medical tool 20 are described in greater detail below that facilitate grasping, puncture, extraction of fluid, delivery of agents, delivery of guidewires, delivery of leads or delivery of other therapy to the pericardium or epicardium of heart 8.

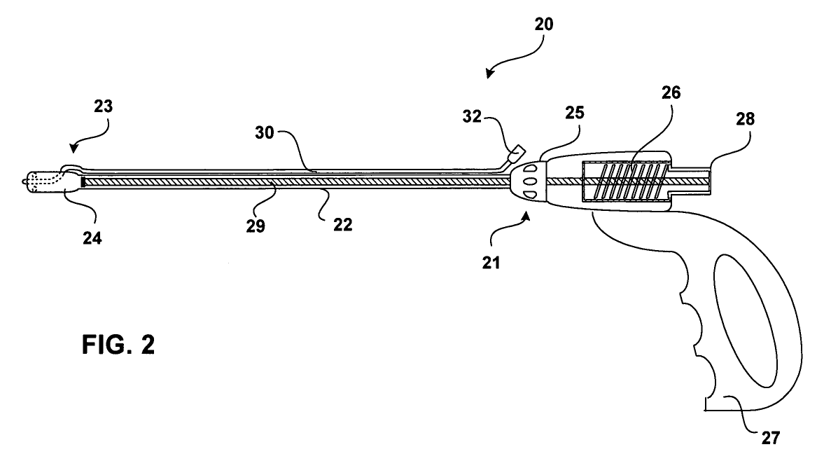

FIG. 2 is a side view of medical tool 20 that can be used for a variety of medical uses, including minimally invasive access to a pericardium. In general, medical tool 20 includes a shaft 22 defining a proximal end 21 and a distal end 23. Jaws 24 for grasping the pericardium of a patient are disposed on the distal end 23 of shaft 22, and a mechanism for opening and closing jaws 24 are connected to proximal end 21 of shaft 22. In this example, a trigger 28 is connected to proximal end 21 of shaft 22 in order to control the opening and closing of jaws 24. For example, jaws 24 may be biased open, and can close upon actuation of trigger 28, e.g., by pressing trigger 28 inward. Trigger 28 can be biased by spring 26, which provides resistance against actuation of trigger 28.

Trigger 28 is mechanically coupled to one or both jaws 24 through shaft 22, e.g., by rod 29 or cable that extends through shaft 22. A physician can hold tool 20 by handle 27 and actuate trigger 28 in order to move jaws 24 between an open position and a closed position. Mechanical coupling between jaws 24 and trigger 28 can also include various levers, hinges, rotating cams, or the like that reside within shaft 22. In the illustrated example, jaws 24 mechanically couple to rod 29 that extends through shaft 22. For example, jaws 24 can be hinged and mechanically coupled to rod 29 that extends through shaft 22. In that case, actuation of trigger 28 pushes rod 29, causing jaws 24 to close about its hinges.

The invention, however, is not limited to such specific mechanical coupling arrangements. Numerous other mechanical arrangements for coupling jaws 24 to a mechanism in order to facilitate movement of jaws 24 could be used, e.g., making use of such mechanical elements as cams, levers, shafts, rods, cables, hinges, or the like. In various embodiments, the mechanism that controls the opening or closing of jaws 24 may comprise a scissor-handle, a trigger, a button, a dial, a slide, a switch, or the like. FIGS. 7-9, discussed in greater detail below, provide additional details of one exemplary mechanical arrangements that can facilitate movement of jaws 24

If desired, a locking element (not shown) can also be provided, e.g., to facilitate locking of jaws 24. Also, a crank 25 can allow for rotation of shaft 22 without rotating handle 27. Accordingly, jaws 24 can be rotated without rotating handle 27, which can help with pericardial grasping in certain instances where patient anatomy requires rotation of jaws 24. If crank 25 is used to allow for rotation of shaft 22 without rotating handle 27, the mechanical arrangement for coupling jaws 24 to trigger 28 would typically not include any rotating cams in order to ensure that rotation of crank 25 did not cause accidental opening or closing of jaws 24.

Medical tool 20 also includes a delivery element 30 positioned parallel and adjacent to shaft 22. A tubing (not shown in FIG. 2), such as a shrink-wrap tubing, can surround delivery element 30 and shaft 22 to secure delivery element 30 to shaft 22. A distal end of delivery element 30 is disposed between jaws 24. For example, delivery element 30 defines a delivery lumen through element 30 that facilitates access to the pericardium of a patient.

The delivery lumen of delivery element 30 can be used for a number of purposes. In particular, a puncture element to puncture the pericardium can be delivered to the pericardium through the delivery lumen of delivery element 30. Also, delivery element 30 can be used to inject agents into the pericardium or to extract fluid from the pericardium. A luer-lock hub 32 or similar structure is formed on a proximal end of delivery element 30 to facilitate attachment to an agent delivery device, such as a syringe, or to facilitate attachment to a fluid extraction device. Also, guide wires or medical leads can be delivered through the delivery lumen of delivery element 30, e.g., for placement on the patient's epicardium. For example, a guidewire can be introduced through delivery element 30 to permit deployment of the medical lead via the guidewire, and then withdrawn following insertion of the lead.

In some cases, a guidewire is introduced through delivery element 30, and a dilator is then delivered over the guidewire. A catheter can then be delivered via the dilator, and a medical lead can be delivered via the catheter. Thus, in that case, the guidewire is withdrawn before lead placement.

In accordance with the invention, jaws 24 of medical tool 20 each include needle-like elements that protrude from jaws 24. Jaws 24 are formed with holes for mating with the needle-like element of the opposing jaw when jaws 24 are closed. In general, sharp needle-like elements facilitate slight puncture and grasping of a patient's pericardium, and can be much more useful for pericardial grasping than coarse ridges or teeth formed on jaws of conventional medical tools. Additional details of the needle-like elements are described below. In the description that follows, each jaw is described as including one needle-like element. However, in general, each jaw may include any number of needle like elements.

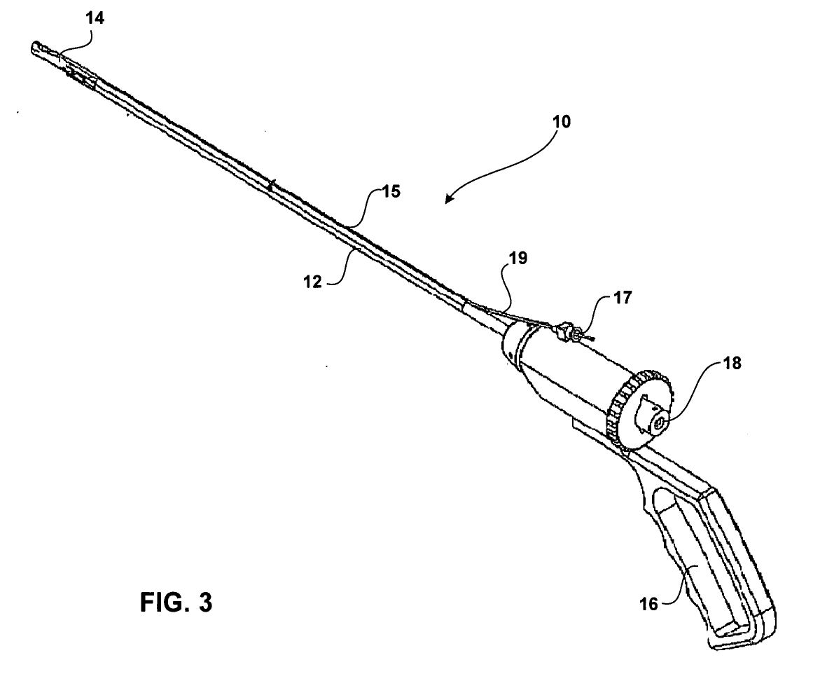

FIG. 3 is a perspective view of medical tool 10 according to another embodiment of the invention. Medical tool 10 includes features similar to those of medical tool 20 of FIG. 2, such as a shaft 12, jaws 14 disposed on the distal end of shaft 12, and a mechanism for opening and closing jaws 14 connected to the proximal end of shaft 12. In the example of FIG. 3 the mechanism for opening and closing jaws 14 comprises trigger 18, although numerous other mechanisms could be used. A handle 16 is also coupled to the second end of shaft 12.

Like medical tool 20 (FIG. 2), medical tool 10 includes a delivery element 19 positioned parallel and adjacent to shaft 12. A shrink-wrap tubing 15 surrounds delivery element 19 and shaft 12 to secure delivery element 19 to shaft 12. A distal end of delivery element 19 is disposed between jaws 14. Delivery element 19 defines a delivery lumen that can be used for a number of purposes including delivery of a puncture needle, delivery of agents, extraction of fluids, delivery of guide wires, delivery of leads, or the like. Delivery element 19 includes a luer-lock hub 17 or similar structure that interfaces with devices such as a syringe, a fluid delivery device, or the like, for injection or withdrawal of fluids.

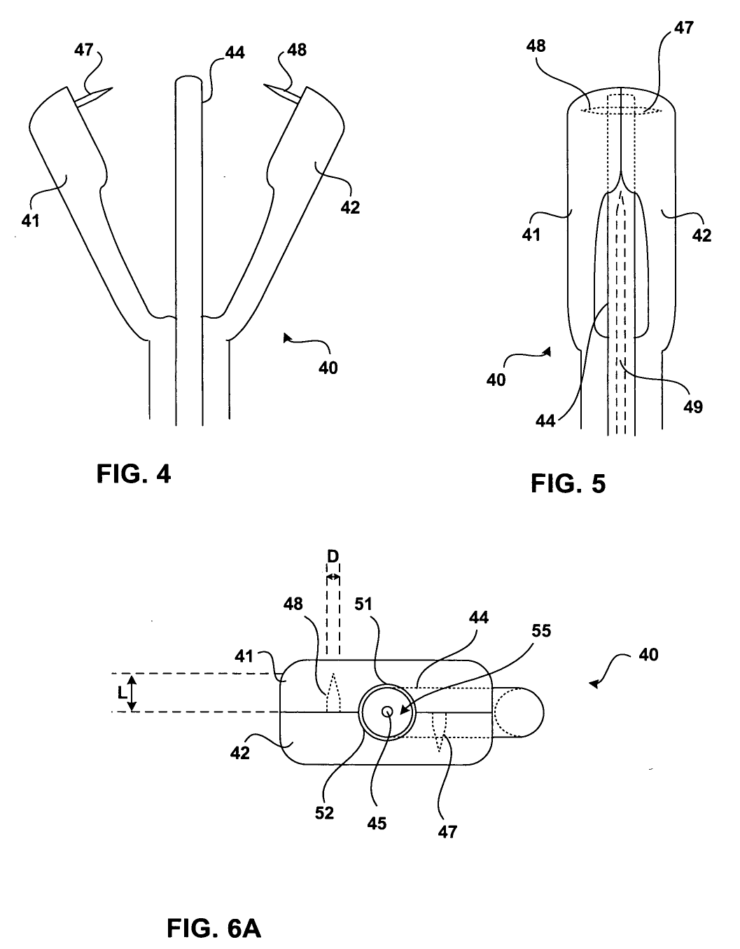

FIGS. 4 and 5 are top views of a distal end of a medical tool 40 that may correspond to tool 10 (FIG. 3) or tool 20 (FIG. 2). As shown in FIGS. 4 and 5, medical tool 40 includes a first jaw 41 and a second jaw 42 that operate in a clamping relationship. The jaws may comprise “Babcock jaws” commonly used in the medical field, with specially designed needle-like elements 47, 48 to facilitate pericardial grasping. First jaw 41 and a second jaw 42 can be opened as shown in FIG. 4 and closed as shown in FIG. 5. In this manner, jaws 41, 42 can clamp onto a pericardium of a patient to facilitate access to the pericardium via delivery element 44. For example, a piercing element 49 may be fed through delivery element 44 in order to pierce a patient's pericardium once the pericardium is grasped by jaws 41, 42. However, numerous other types of structures or elements could alternatively be fed through deliver element 44.

First jaw 41 includes a first needle-like element 47 and second jaw includes a second needle-like element 48. Any number of needle-like elements can be disposed on each jaw. Each of needle-like elements 47, 48 typically comprises an elongated cylindrical structure with a sharpened distal tip. Alternatively, needle-like elements 47, 48 could have a square-shaped cross-section or any other cross-sectional shape. In any case, needle-like elements 47, 48 are particularly suited to improve pericardial grasping by jaws 41, 42. In particular, when jaws 41, 42 are clamped onto a patient's pericardium, needle-like elements 47, 48 pierce through the pericardium so that tool 40 can be used to pull the pericardium from the heart (sometimes referred to as “tenting”). Other uses of tool 40 include removal of fat or grasping of any membranous tissue that is similar to the pericardium.

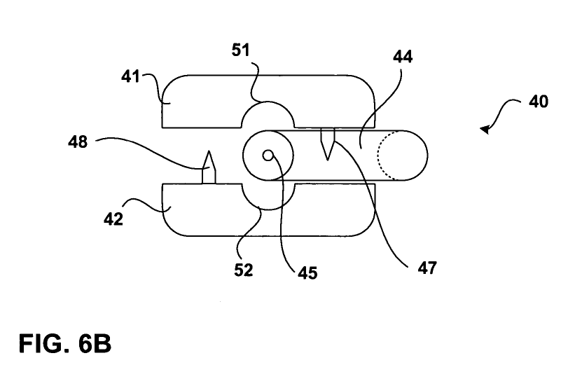

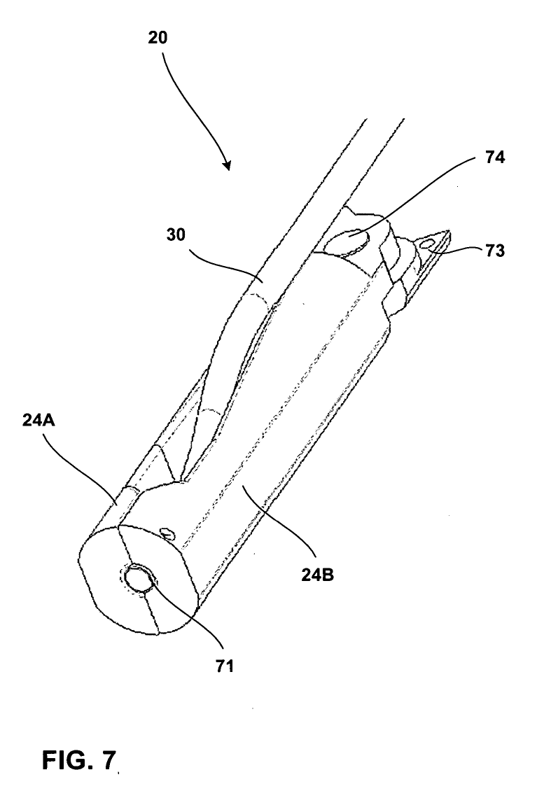

The most desirable size of needle-like elements 47, 48 can vary for different patients, e.g., depending on the amount of fat tissue in proximity to the pericardium. Thus, upon inspection of a patient's pericardium, e.g., via endoscopy, it can be desirable to select the size of needle-like elements 47, 48 in order to ensure that adequate pericardial grasping can be archived. For example, a physician can select from different tools having different sized needle-like elements. Alternatively, needle-like elements 47, 48 or jaws 41, 42 can be removable such that a physician can select desired jaws or needle-like elements, and attach the selected jaws or elements to tool 40. Exemplary desirable dimensions of needle-like elements 47, 48 include cylindrical diameters (D) in the range of 0.005 inch to 0.140 inch and lengths (L) of 0.020 inch to 0.300 inch (see dimensions labeled in FIG. 6), where the length refers to the distance from a surface of one of jaws 41, 42 to a distal tip of the corresponding needle-like element 47 or 48.

Once the pericardium has been grasped by tool 40, delivery element 44 can be used to deliver agents, structures, or therapy to a patient through the pericardium. Also, delivery element 44 can be used to extract pericardial fluid for diagnosis purposes. Delivery element 44 defines a delivery lumen that facilities delivery of agents or structures, or extraction of pericardial fluid. By way of example, piercing element 49 is illustrated in FIG. 5 within the delivery lumen of delivery element 44.

FIGS. 6A and 6B are front views of a distal tip of tool 40. In particular, FIG. 6A depicts tool 40 when jaws 41, 42 are closed as shown in FIG. 5 and FIG. 6B depicts tool 40 when jaws 41, 42 are open as shown in FIG. 4. First jaw 41 is formed with a hole for mating with needle-like element 48 of second jaw 42 when jaws 41, 42 are closed. Similarly, second jaw 42 is formed with a hole for mating with needle-like element 47 of second jaw when jaws 41, 42 are closed. The distal end of each of the first and second jaws 41, 42 defines a semi-circular shape 51, 52, such that when first and second jaws 41, 42 are closed, a hole 55 is formed by semi-circular shapes 51, 52. In other words, semi-circular shapes 51, 52 comprise channels or grooves formed along inner surfaces of jaws 51, 52. Delivery element 44 is disposed along the shaft of tool 40 and passes through hole 55 at the distal end of tool 40 for access to a patient's pericardium, or other tissue. Accordingly, therapy can be delivered to a patient's pericardium or other tissue via a delivery lumen 45 formed in delivery element 44. Such therapy may include delivery of fluids, gene therapy, delivery of a lead, and the like.

Referring again to FIG. 2, the parallel and adjacent positioning of delivery element 30 relative to shaft 22 allows for additional design flexibility with respect to the interior of shaft 22. For example, mechanical components used to facilitate opening and closing of jaws 24 typically reside at least partially within shaft 22. Moreover, in some cases, an additional elements may feed through shaft 22, such as another delivery element, a piercing needle, a sensor, a suction lumen, or possibly an endoscope used to view the patients pericardium while the pericardium is being grasped by tool 20. In any case, because delivery element 30 is not positioned within shaft 22, additional design flexibility exists for medical tool 20.

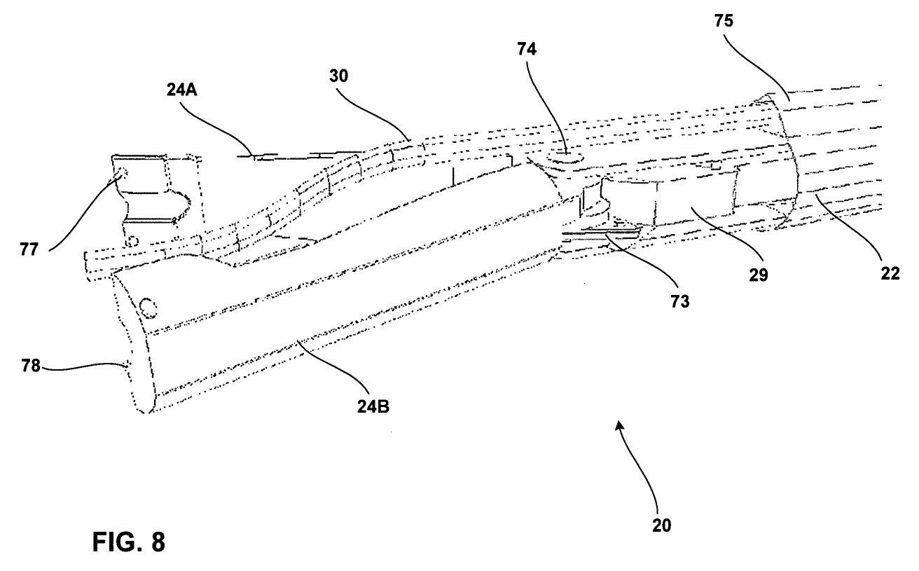

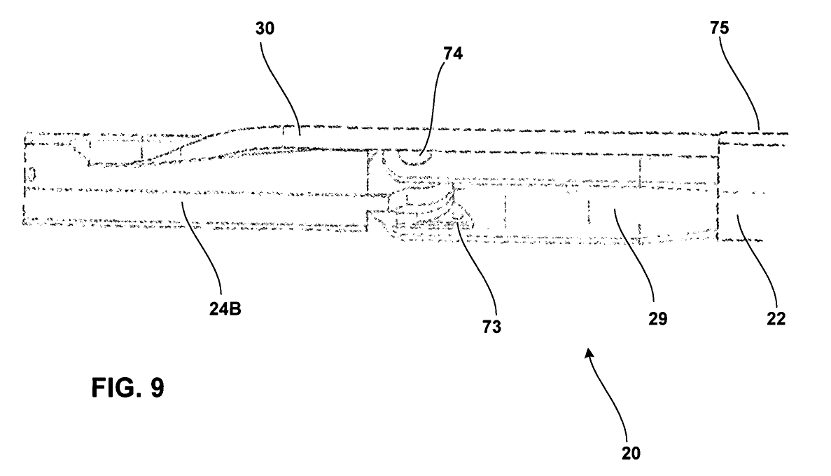

FIGS. 7-9 are perspective views of distal end 23 of medical tool 20 illustrated in FIG. 2. As shown in FIGS. 7-9, medical tool 20 includes a first jaw 24A and a second jaw 24B that operate in a clamping relationship. Moreover jaws 24 include specially designed needle-like elements 77, 78 (FIG. 8) to facilitate pericardial grasping. First jaw 24A and a second jaw 24B can be opened as shown in FIG. 8 and closed as shown in FIGS. 7 and 9. A delivery element 30 extends parallel to shaft 22 as is bent to feed through jaws 24. A shrink wrap tubing 75 secures delivery element 30 to shaft 22. A hole 71 (FIG. 7) is formed at the distal end of jaws 24, when closed, such that delivery element 30 provides access to grasped tissue through jaws 24.

In order to facilitate opening and closing of jaws 24, a rod 29 or other mechanical element, extends through shaft 22. When a physician actuates trigger 28, rod 29 moves towards jaws 24. This movement of rod 29 causes rod 29 to push lever 73 and a corresponding lever on the opposing side of lever 73. Jaw 24A is mechanically coupled to lever 73 and jaw 24B is likewise coupled to the other lever on the opposing side of levers 73. When rod pushes against the levers, jaws 24 are caused to open about hinge 74. In this manner, actuation trigger 28 causes the opening of jaws 24. Spring 26 (FIG. 2) may bias trigger 28 so that jaws 24 remain biased closed until trigger 28 is pushed inward. The mechanical arrangement of FIGS. 7-9 is exemplary, and numerous other mechanical arrangements could also be used to facilitate opening and closing of jaws 24.

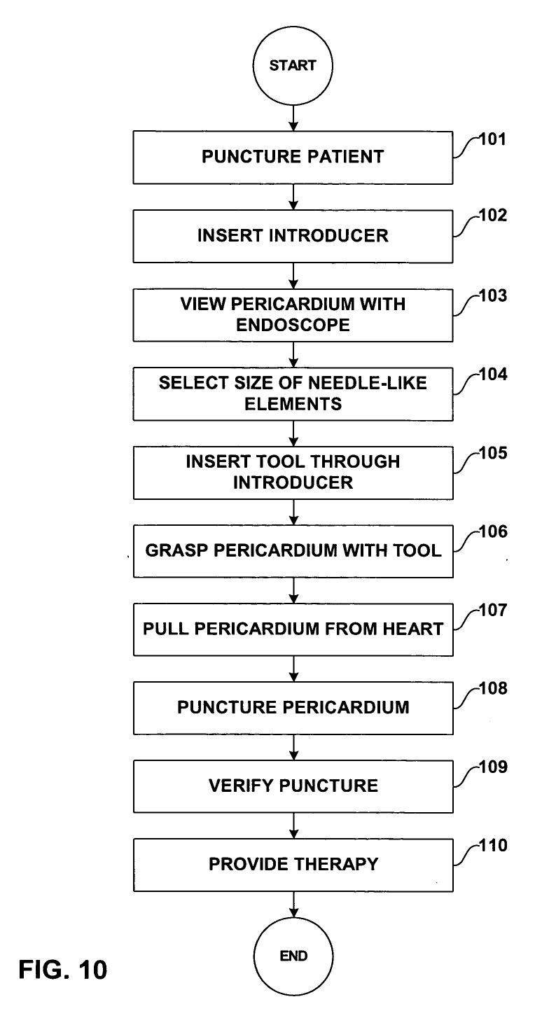

FIG. 10 is an exemplary flow diagram according to an embodiment of the invention, illustrating a medical procedure to provide therapy to a pericardium of a patient. The technique illustrated in FIG. 10 can be implemented by a physician making use of a medical tool having one of more of the features illustrated in FIGS. 1-9. FIG. 10 will be explained with reference to medical tool 40 of FIGS. 4-6B, which as mentioned above, may correspond to medical tool 20 or medical tool 10 of FIGS. 2 and 3 respectively.

As shown in FIG. 10, a physician punctures a patient (101), and inserts an introducer, such as a guiding catheter, to close proximity to the patients heart (102). For example, the physician could puncture through the patient's diaphragm, or the patient's ribs for access to the patients heart. In any case, the physician then inserts an endoscope through the introducer to view the patient's pericardium (103). Based on the appearance of the patient's pericardium, the physician selects a size of needle-like elements 47, 48 of a medial tool 40 (104). In particular, the physician can select a length that will perforate the pericardium once it has been firmly grasped, without choosing a length that is too long to cause a risk of damaging the epicardium. For example, differing sized needle-like elements can be more useful for pericardial grasping depending on the amount of fat tissue or other anatomy associated with the patient's pericardium. The physician can select from a plurality of tools having differing sized needle-like elements, or can select specific jaws or needle-like elements to be attached to the distal end of shaft 42.

Once the desired configuration is obtained, the physician inserts the tool through the introducer for access to the patient's pericardium (105). Using the tool, the physician grasps the patients pericardium (106). Needle-like elements 47, 48 disposed on jaws 41, 42 can be particularly useful for pericardial grasping, relative to ridges or teeth formed on jaws of conventional medical tools. Such needle-like elements 47, 48 puncture the pericardium to facilitate the grasping by jaws 41, 42. The physician actuates a mechanism such as trigger 28 (FIG. 2) to cause jaws 41, 42 to clamp onto the pericardium and needle-like elements 47, 48 to pierce the pericardium.

Once tool 40 has grasped the pericardium, the physician pulls pericardium from the heart (sometimes referred to as “tenting”) (107) and punctures the pericardium using tool 40 (108). For example, a puncture element can be extended through a delivery lumen 45 of delivery element 44 in order to puncture the pericardium. In that case, the physician selects a desirable sized puncture element based on the prior endoscope view of the pericardium. Alternatively, delivery element 44 can be extended through hole 55 to perform the puncture. In any case, once the pericardium has been punctured, the physician verifies the puncture (89), e.g., by extracting pericardial fluid or injecting dye. The physician can then provide therapy to the patient via the delivery lumen 45 of delivery element 44. Such therapy may include delivery of agents, extraction of fluids, delivery of guide wires, delivery of leads, or the like.

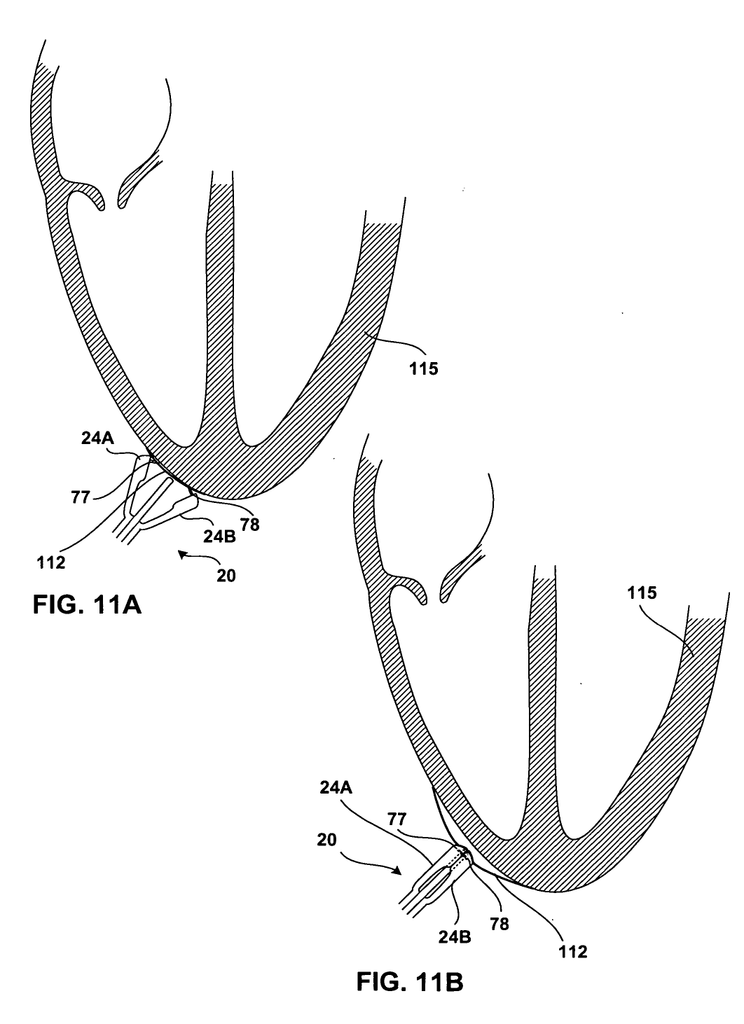

FIGS. 11A and 11B are conceptual views of a distal end of medial tool 20 grasping the pericardium 112 of heart 115 in accordance with an embodiment of the invention. Needle-like elements 77, 78 specifically facilitate pericardial grasping in an improved fashion relative to conventional tools that include non-needle like teeth. First jaw 24A and second jaw 24B can be opened as shown in FIG. 11A. Needle-like elements 77, 78 pierce pericardium 112 as jaws 24 are closed. Once jaws 24 are closed as shown in FIG. 11B, needle-like elements 77, 78 are pierced through pericardium 112 such that a good grasp of pericardium 112 is achieved by jaws 24. Accordingly, pericardium 112 can be pulled from heart 115 so that drugs, agents or therapy can be delivered to the pericardial space or extraction of fluid can occur.

A number of embodiments of the invention have been described. In particular, a medical tool has been described that can facilitate minimally invasive access to the pericardium. A number of desirable features of the medical tool have been described including jaws that include needle-like elements that extend from the jaws, and a delivery element defining a delivery lumen that extends along the tool, parallel and adjacent to a shaft, with a distal end of the delivery element being positioned between the jaws. Such features can improve pericardial grasping, puncture and therapy delivery via the tool. Nevertheless, various modifications can be made to the description above without departing from the scope of the following claims. For example, the described medical tool can be used for numerous other purposes including non-pericardial uses and uses with more invasive medical procedures such as open-chest surgery. In particular, needle-like elements can facilitate improved grasping of any internal membranous structure or tissue. These and other embodiments are within the scope of the following claims.

1. A medical tool comprising:
a shaft defining a proximal end and a distal end;
first and second jaws disposed on the distal end of the shaft:
a first needle-like element protruding from the first jaw;
a second needle-like element protruding from the second jaw;
a delivery element defining a delivery lumen and positioned parallel and adjacent to the shaft with a distal end of the delivery element being disposed between the first and second jaws; and
a mechanism coupled to the proximal end of the shaft and mechanically coupled to the first and second jaws to open and close the first and second jaws.

2. The medical tool of claim 1, wherein the delivery lumen of the delivery element defines a path for delivery of agents through the delivery element or extraction of fluid through the delivery element.

3. The medical tool of claim 1, wherein a proximal end of the delivery element includes a hub for coupling the delivery element to another device.

4. The medical tool of claim 3, wherein the hub is sized for mechanical coupling to an agent delivery device.

5. The medical tool of claim 3, wherein the hub is sized for mechanical coupling to a fluid extraction device.

6. The medical tool of claim 1, wherein the first jaw defines a first hole for mating with the second needle-like element of the second jaw and the second jaw defines a second hole for mating with the first needle-like element of the first jaw.

7. The medical tool of claim 1, wherein a distal end of each of the first and second jaws defines a semicircular shape such that when the first and second jaws are closed, a hole through the closed jaws Is formed by the semi-circular shapes.

8. The medical tool of claim 7, wherein a distal end of the delivery element passes through the hole.

9. The medical tool of claim 1, wherein the mechanism to open and close the first and second jaws comprises a trigger coupled to the jaws through the shaft.

10. The medical tool of claim 1, wherein the mechanism to open and close the first and second jaws comprises a button coupled to the jaws through the shaft.

11. The medical tool of claim 1, further comprising a handle coupled to the second end of the shaft.

12. The medical tool of claim 1, wherein the shaft is rotatable relative to the handle to rotate the jaws without rotating the handle.

13. The medical tool of claim 1, wherein the medical tool comprises a pericardial access tool.

14. The medical tool of claim 1, further comprising a tubing around the delivery element and the shaft to secure the delivery element to the shaft.

15. The medical tool of claim 1, further comprising a locking element to lock and unlock the first and second jaws.

16. The medical tool of claim 1, wherein the shaft is formed with a lumen.

17. The medical tool of claim 1, further comprising a puncture element positioned through the delivery lumen of the delivery element.

18. A pericardial access tool comprising:
a shaft defining a proximal end and a distal end;
first and second jaws disposed on the distal end of the shaft:
first and second needlelike elements protruding respectively from the first and second jaws for puncturing a pericardium so that the first and second jaws can grasp the pericardium;
a delivery means positioned parallel and adjacent to the shaft with a distal end of the delivery means being disposed between the first and second jaws for insertion into the pericardium when the pericardium is grasped by the first and second jaws; and
a mechanism coupled to the proximal end of the shaft and mechanically coupled to the first and second jaws to open and close the first and second jaws to grasp the pericardium.

19. The pericardial access tool of claim 18, wherein the first and second jaws are formed to collectively define a hole on a distal end of the jaws when the jaws are closed, and wherein the distal end of the delivery means passes through the hole.

20. The pericardial access tool of claim 18, further comprising a puncture means positioned within a lumen of the delivery means to puncture the pericardium when the pericardium is grasp by the first and second jaws.

21. A method comprising:
inserting a pericardial access tool Into a patient, the pericardial access tool including needle-like element disposed on Jaws of the tool; and
grasping a pericardium of the patient with the jaws of the tool.

22. The method of claim 21, further comprising selecting a size of needle-like elements of the pericardial access tool based on inspection of the pericardium.

23. The method of claim 21, further comprising puncturing the pericardium with a puncture element of the pericardial access tool, wherein the puncture element passes through a lumen of a delivery element of the tool and the delivery element passes through a hole formed on a distal end of the jaws.

24. The method of claim 23, further comprising selecting the puncture element based on inspection of the pericardium.

25. The method of claim 23, further comprising extracting fluid from the pericardium through the lumen of the delivery element.

26. The method of claim 23, further comprising injecting an agent into the pericardium through the lumen of the delivery element.

27. The method of claim 23, further comprising inserting a medical lead through the lumen of the delivery element to position the medial lead in proximity to an epicardium of the patient.

28. The method of claim 23, further comprising Inserting a guide wire through the lumen of the delivery element and inserting a medical lead over the guide wire to position the medial lead in proximity to an epicardium of the patient.

29. The method of claim 21, further comprising puncturing the patient and inserting an introducer, wherein the pericardial access tool is inserted through the introducer.
